(19) United States
(12) Patent Application Publication
(10) Pub. No.: US 2004/0024439 A1
(43) Pub. Date: Feb. 5, 2004

(54) Nerve cuff electrode

(76) Inventor: Ronald R. Riso, Aalborg (DK)

Correspondence Address: Barbara E Johnson, 700 Koppers Building, 436 Seventh Avenue, Pittsburgh, PA, 15219-1818, US

(21) Appl. No.: 10/398,846

(22) PCT Filed: Oct. 11, 2001
(86) PCT No.: PCT/DK01/00666

(30) Foreign Application Priority Data
Oct. 11, 2000 (DK) PA 200001513

(51) Int. Cl. A61N 1/05

(52) U.S. Cl. 607/117; 607/116

(57) ABSTRACT

A nerve electrode system for stimulating and/or monitoring at least one nerve fascicle in a trunk nerve comprising at least one internal electrode and at least one external electrode. The invention also relates to a multi-polar nerve cuff, a method of installing a nerve electrode system or a multi-polar nerve cuff and finally the invention relates to uses of the nerve electrode system or the multi-polar nerve cuff.

### FIELD OF THE INVENTION

The invention relates to a nerve electrode system for stimulating and/or monitoring at least one nerve fascicle in a trunk nerve according to the preamble of claim 1, a multi-polar nerve cuff for stimulating and/or monitoring at least one nerve fascicle in a trunk nerve according to the preamble of claim 10, a method of installing a nerve electrode system or a multi-polar nerve cuff according to the preamble of claim 18 and uses according to claims 19-21.

### BACKGROUND OF THE INVENTION

Electrical stimulation of nerves, nerve fibers etc, has been intensively studied in an effort to e.g. activate muscles. In this context nerve cuff electrodes are being used for applying electrical stimulation to peripheral nerves. Such prior art nerve cuffs are wrapped around the nerve and are provided with a number of discrete electrodes in the form of surface electrodes that can contact the nerve, i.e. the surface of the peripheral nerve trunk at different positions.

By means of these prior art nerve cuffs it is possible, by applying electrical signals to different electrodes, e.g. pair of electrodes, tri-polar combinations etc. to stimulate or activate different nerve fascicles in the nerve trunk, whereby muscles may be activated. However, this works best for underlying fascicles lying near the surface of the trunk nerve. It has not been possible with these prior art arrangements to selectively activate fascicles that are deep within the trunk nerve without also activating the fascicles that lie near to the surface.

Therefore, even though such prior art nerve cuffs have been provided with several discrete contact sites, e.g. four sets of tripoles distributed circumferentially around the nerve trunk, it may be difficult or even impossible to perform a selective stimulation, e.g. a stimulation of a single nerve fascicle, a portion of a nerve fascicle or of specific nerve fascicles giving a desired muscle reaction without unintentionally activating other nerve fascicles and/or muscles.

Other techniques for applying electrical stimuli to individual nerve fascicles have been studied in the past, e.g. the implantation of wires or other conductors into the interior of a trunk nerve. However these techniques all suffer from the drawback that a number of electrodes have to be implanted in order to achieve some degree of selectivity and that in general only the nerve fascicles associated with a contact site may be activated.

Further U.S. Pat. No. 5,400,784 describes a slowly penetrating inter-fascicular cuff electrode that comprises a number of electrode sets arranged circumferentially with 90° spacing. These electrode sets are arranged in such a manner that the electrodes are able to penetrate the trunk nerve in order to provide a number of interfascicular electrodes, i.e. electrodes arranged in the interior of the nerve trunk in the space between the individual nerve fascicles. This prior art cuff electrode, which is specifically designed to avoid damage to the individual nerve fascicles, e.g. the perineurium membrane surrounding the nerve fascicles is not penetrated, will be able to provide stimulation of nerve fascicles in the interior of the trunk nerve.

However, it will normally not be possible to stimulate individual nerve fascicles with this prior art nerve cuff since the number of electrode contacts sets is limited and since the stimulation which may be applied by two sets of electrodes may potentially be applied to all nerve fascicles present in a compartment between two sets of electrodes.

### SUMMARY OF THE INVENTION

One of the objectives of the invention is thus to provide a nerve cuff electrode by means of which the ability to achieve selective and graded activation or stimulation of a nerve fascicle or nerve fascicules is improved.

A further objective is to provide a nerve cuff electrode by means of which the ability to achieve selective and graded activation or stimulation of nerve fascicules that are positioned deep within the interior of a multi-fascicle nerve is improved.

Further, an objective of the invention is to achieve activation or stimulation of nerve fascicules that are positioned deep within the interior of a multi-fascicle nerve without unintentionally activating other nerve fascicles.

These and other objectives are achieved by the invention as explained in the following.

As specified in claim 1, the invention relates to a nerve electrode system for stimulating and/or monitoring at least one nerve fascicle in a trunk nerve comprising at least one internal electrode and at least one external electrode arranged in a cuff carrier system.

Hereby it is achieved that individual nerve fascicles may be selected with a high degree of accuracy since the electrical fields applied or monitored may be located in directions and in ranges from a point located in the interior of a nerve trunk to a point on the surface of the nerve trunk.

Hereby, it will also be possible to provide a selective and graded activation or a selective monitoring of a nerve fascicle or nerve fascicles that lie within and even deep within the nerve trunk. An added advantage is that this is achieved without having to implant a relatively large number of electrodes in the interior of the nerve trunk itself or even in the interior of individual nerve fascicles, since an improvement may be achieved by the invention by implanting only one internal electrode. Thereby the extent of the surgical work, which must be performed when applying the electrode system and in particular the internal electrode/electrodes will be minimized which is advantageous, particularly since the surgical work may be associated with a potential risk of provoking nerve damage.

It shall be mentioned here that by the term “external electrode” shall in this context be understood any type of electrode that is placed on the surface of a trunk nerve or in the vicinity of said surface.

Similarly is shall be stated that by the term “internal electrode” shall in this context be understood any type of electrode that is at least partly placed in the interior of a trunk nerve (below said surface).

Preferably, as specified in claim 2, said at least one internal electrode may be an intrafascicular electrode, whereby it is achieved that an intimate connection with a specific nerve fascicle may be achieved, and simultaneously the possibility of stimulating or monitoring other nerve fascicles by means of said intrafascicular electrode in combination with external electrodes is maintained.

According to the embodiment characterized in claim 3, said at least one internal electrode may be an interfascicular electrode, whereby it is achieved that the at least one internal electrode can be introduced, e.g. implanted, in the interior of the trunk nerve in a relatively uncomplicated manner and simultaneously a high degree of selectivity may never the less be obtained.

Preferably, as stated in claim 4, said at least one external electrode is a circumferential electrode, whereby this or these electrodes may be applied to the trunk nerve in an advantageous manner.

According to the embodiment characterized in claim 5 the nerve electrode system may comprise a number of external electrodes, placed spatially along the length and/or along the circumference of a trunk nerve, whereby a high degree of selectivity may be obtained. Since in this manner the number of external electrodes may be chosen without special considerations to available space, which for example must be born in mind when specifying e.g. the number of contact sites or electrodes to be placed in the interior of a trunk nerve, the number of e.g. bipolar contact sets to be selected for stimulation or monitoring may be relatively high, which is achieved in an advantageous manner.

Preferably, said at least one external electrode is located on a base part for said system, whereby an advantageous structure for the system, e.g. a structure forming means for connecting and holding the parts of the system is achieved.

Advantageously, said base part may be in the form of a cuff designed to be placed on a trunk nerve, whereby the system according to the invention may be applied to and located on a trunk nerve with relatively simple means.

According to the embodiment specified in claim 6, said at least one internal electrode, preferably in the form of an intra- or interfascicular electrode, or a connection for said internal electrode, may be connected to a base part of said cuff carrier system, whereby an advantageous manner of arranging the necessary conducting leads is achieved. Further, it is achieved that mechanically all electrodes of the system might be interconnected.

However, it shall be pointed out that such a connection to the base part need not be present in order to achieve the objectives of the invention.

Preferably, as stated in claim 7, said at least one internal electrode, preferably in the form of an intra- or interfascicular electrode, may comprise a conductor, e.g. in the form of a wire, a filament and/or a ribbon made of metal, electrically conducting polymer, and/or another electrically conducting material.

As stated in claim 8, said at least one internal electrode may comprise a carrier for said conductor, e.g. in the form of a polymer, polymer ribbons overlayed with conductive traces or other electrode carriers.

In a further preferred embodiment characterized in claim 9, said at least one internal electrode may comprise means for insulation and said insulation may locally be absent or may locally be removed to provide a contact site. In this manner a suitable contact site may be provided in a simple and efficient manner.

Further, the invention relates to a multi-polar nerve cuff for stimulating and/or monitoring at least one nerve fascicle in a trunk nerve as stated in claim 10, which according to the invention comprises at least one internal electrode for providing an electrode site within the trunk nerve.

Hereby a multi-polar nerve cuff is provided by means of which individual nerve fascicles may be selected with a high degree of accuracy since the electrical fields applied or monitored may be located in directions and in ranges from a point located in the interior of a nerve trunk to a point on the surface of the nerve trunk.

Hereby, by means of the nerve cuff according to the invention it will also be possible to provide a selective and graded activation or monitoring of a nerve fascicle or nerve fascicles that lie within and even deep within the nerve trunk. An added advantage is that this is achieved without having to implant a relatively large number of electrodes in the interior of the nerve trunk itself or even in the interior of individual nerve fascicles, since an improvement may be achieved by the invention by implanting only one internal electrode. Thereby the extent of the surgical work, which must be performed when applying the multi-polar nerve cuff and in particular the internal electrode/electrodes will be minimized which is advantageous, particularly since the surgical work may be associated with a potential risk of provoking nerve damage.

Preferably, as stated in claim 11, said at least one internal electrode may be an intrafascicular electrode, whereby a nerve cuff is provided by means of which it is achieved that an intimate connection with a specific nerve fascicle may be obtained, and simultaneously the possibility of stimulating or monitoring other nerve fascicles by means of said intrafascicular electrode in combination with external electrodes is maintained.

According to the embodiment characterized in claim 12, said at least one internal electrode may be an interfascicular electrode, whereby it is achieved that the at least one internal electrode can be introduced, e.g. implanted, in the interior of the trunk nerve in a relatively uncomplicated manner and simultaneously a high degree of selectivity may never the less be obtained.

According to the embodiment characterized in claim 13, the multi-polar nerve cuff may comprise a number of circumferential electrodes, spatially placed along the length and/or along the circumference of the nerve cuff, whereby a high degree of selectivity may be obtained. Since in this manner the number of external electrodes may be chosen without special considerations to available space, which for example must be born in mind when specifying e.g. the number of contact sites or electrodes to be placed in the interior of a trunk nerve, the number of e.g. bipolar contact sets to be selected for stimulation or monitoring may be relatively high, which is achieved in an advantageous manner.

According to the embodiment specified in claim 14, said at least one internal electrode, preferably in the form of an intra- or interfascicular electrode, or a connection for said internal electrode, may be connected to a base part of the nerve cuff, whereby an advantageous manner of arranging the necessary conducting leads in the multi-polar nerve cuff is achieved. Further, it is achieved that mechanically all electrodes of the nerve cuff will be interconnected.

However, it shall be pointed out that such a connection to the base part need not be present in order to achieve the objectives of the invention.

Preferably, as stated in claim 15, said at least one internal electrode, preferably in the form of an intra- or interfascicular electrode, may comprise a conductor, e.g. in the form of a wire, a filament and/or a ribbon made of metal, electrically conducting polymer, and/or another electrically conducting material.

Advantageously, as stated in claim 16, said at least one internal electrode may comprise a carrier for said conductor, e.g. in the form a polymer, polymer ribbons overlayed with conductive traces or other electrode carriers.

In a further preferred embodiment as specified in claim 17, said at least one internal electrode may comprise means for insulation and said insulation may locally be absent or may locally be removed to provide a contact site. In this manner a suitable contact site may be provided in a simple and efficient manner according to the invention.

Further, the invention relates to, as specified in claim 18, a method of installing a nerve electrode system according to one or more of claims 1-9 or a multi-polar nerve cuff according to one or more of claims 10-17 whereby

at least one internal electrode is introduced in a trunk nerve, after which

a basis part, e.g. a cuff of said system or nerve cuff is applied around the trunk nerve in the vicinity of the internal electrode, or vice versa.

Hereby, it is achieved that the electrode system and/or the nerve cuff according to the invention may be applied to a nerve trunk in an advantageous manner. The electrode system and/or the nerve cuff will of course normally have to be applied to a nerve trunk, e.g. a peripheral nerve trunk of a human being by means of some sort of surgical process. The extent of the surgical process will be minimized by the method according to the invention, and thus also the risk of inducing potential problems, damages etc. The implantation of the internal electrodes, e.g. in the interior of the nerve trunk or even in the interior of an individual nerve fascicle, may be performed in connection with the implantation of the nerve cuff. Further, by this method, the nerve cuff will be placed in the vicinity of the contact site(s) of the internal electrode(s), whereby the nerve cuff will have a protective effect and whereby conductive leads etc. from internal and external electrodes may be comprised in a common cable.

Finally, the invention relates to uses of said nerve electrode system and/or said multipolar nerve cuff.

As stated in claim 19, the invention relates to a use of a nerve electrode system according to one or more of claims 1-9 or a multi-polar nerve cuff according to one or more of claims 10-17 for selective and graded activation or stimulation of nerve fascicles, e.g. motor nerve fascicles.

Hereby the invention may be utilized for restoring muscle contractions for persons, e.g. for the restoration of movement for persons that have muscle paralysis caused by e.g. head trauma, stroke, spinal cord injury etc. The restored motor functions may include grasping objects, standing and wallding, bladder or bowel functions etc.

As stated in claim 20, the invention relates to the use of a nerve electrode system according to one or more of claims 1-9 or a multi-polar nerve cuff according to one or more of claims 10-17 for selective and graded activation or stimulation of nerves such as peripheral nerves, cranial nerves, spinal nerves etc. for restoration of sensory functions, e.g. activation or stimulation of optic nerves, auditory nerves or other sensory nerves.

Hereby the invention may be utilized for the electrical selective and graded activation of nerves such as peripheral nerves, cranial nerves, spinal nerves etc involving sensory functions. Hereby for example, activation or stimulation of the optic nerves can help to restore vision in some blind individuals, activation or stimulation of the auditory nerves can partially restore hearing, activation or stimulation of sensory nerve fibers in the residual limb of amputees can be used to input cognitive sensory feedback for prosthesis users, etc.

As stated in claim 21, the invention relates to a use of a nerve electrode system according to one or more of claims 1-9 or a multi-polar nerve cuff according to one or more of claims 10-17 for recording or monitoring of nerve fascicles, e.g. for neuroprosthesis control etc.

Hereby the invention may be utilized for monitoring, e.g. to record nerve activity signals for e.g. the purpose of obtaining sensory feedback signals for closed loop neuroprosthesis control and for obtaining command signals for the control of artificial limbs. The use of the nerve electrode system and/or the multi-polar nerve cuff according to the invention will provide an improved performance, which will be of significant importance in these applications.

### THE FIGURES

The invention will be described in further detail below with reference to the drawings of which

FIG. 1 shows in a perspective view a nerve electrode system with a multi-polar cuff according to an embodiment of the invention applied to a trunk nerve,

FIG. 2 is a perspective view of a multi-polar nerve cuff which may be utilized in connection with the invention,

FIG. 3 shows activation profiles of muscles of an animal when using prior art nerve cuff contacts for stimulation, and

FIG. 4 shows corresponding activation profiles when using a nerve cuff electrode according to the invention, e.g. with an interfascicular contact.

### DETAILED DESCRIPTION

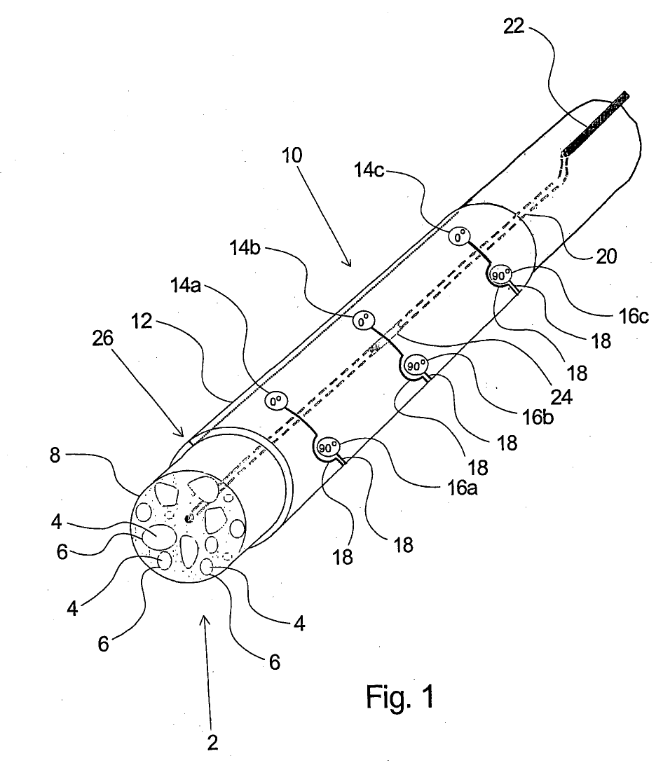

A nerve electrode system and a multi-polar nerve cuff according to an embodiment of the invention will be described with reference to FIG. 1. In this figure the application of the invention to a multi-fascicle nerve has been illustrated. The multi-fascicle nerve, also referred to as a trunk nerve or a peripheral nerve, is generally designated 2 and comprises a number of nerve fascicles 4. These nerve fascicles 4 each comprises a number of individual nerve fibers (not shown) and are each provided with a surrounding membrane 6 called perineunum. Further, an outer membrane 8 called epineurium surrounds the bundle of nerve fascicles 4 comprised in the peripheral nerve 2.

FIG. 1 illustrates a multi-polar nerve cuff generally designated 10, which has been installed on the trunk nerve 2. The nerve cuff 10 comprises a basis part 12 which is shaped as a cuff device and which is wrapped around the trunk nerve 2. The basis or cuff part 12 comprises a number of external electrodes, which are placed on the interior wall of the basis or cuff part 12. In FIG. 1 six of these electrodes are illustrated, i.e. two tripolar sets, distributed circumferentially around the trunk nerve 2. One of these tripolar sets comprises the contacts 14a, 14b and 14c and is located at a 0° position and the other one comprises the contacts 16a, 16b and 16c and is located at a 90° position. Two further tripolar sets of contact electrodes (not visible in FIG. 1) are placed on the basis or cuff part 12 at 180° and at 270°, respectively. As shown, each set of electrodes is placed longitudinally along the basis or cuff part 12 with the contact sites evenly distributed in an axial direction. Further, a conductor 18, e.g. a conductor trace located on the basis or cuff part 12 is connected to each contact electrode for transmission of monitoring and/or stimulating signals between the electrodes and external circuitry, processing means, control circuitry or other similar means.

The electrodes 14a-c and 16a-c are located as described above on the inner side of the basis or cuff part 12, e.g. located on an interior wall of the base part 12. This part can consist of a planar structure, which is wrapped around the nerve so that is has a coiled or spiralling form. Further, the cuff part can be designed to be a self-coiling structure. Instead the cuff part can be of a tubular structure which is provided with an axial slit or opening 26 so that the cuff can be placed around the trunk nerve. The basis or cuff part may be flexible whereby it will grip the nerve trunk and whereby the contact electrodes 14a-c and 16a-c etc. will be positioned in contact with the exterior of the trunk nerve 2 at different positions, i.e. in contact with the epineurium 8 of the trunk nerve 2.

An internal electrode 20 is also shown in FIG. 1. This electrode has been introduced in the trunk nerve 2 as illustrated by broken lines, for example in the middle of the trunk nerve or near the middle. As shown a lead-in or connection 22 for this electrode is provided leading to/from the outside of the trunk nerve and providing connection to the necessary circuitry etc. An internal contact site 24 has been established, e.g. by removing part of the insulation of the internal electrode 20, by providing a window in the insulation or in other suitable ways whereby a contact site for a nerve fascicle located in the inner part of the trunk nerve may be established.

This internal contact site may be established as an intrafascicular contact site, e.g. a contact site established in a nerve fascicle 4 comprising a number of individual nerve fibers. Such a contact site may be established by perforating the perineurium 6 and introducing the internal electrode to the inner compartment of the nerve fascicle 4 for a sufficient length.

The internal contact site may alternatively be established as an interfascicular contact site, e.g. a contact site established (deep) in the interior of a trunk nerve 2 comprising a number of nerve fascicles 4, located in a space between these nerve fascicles 4.

In FIG. 1 only one internal electrode is shown. However, it is possible to provide the system according to the invention with more than one internal electrode or contact, e.g. two, three, four or even more may be utilised. Each of these may establish an intrafascicular contact site or an interfascicular contact site. whereby a suitable combination may be established in accordance with the particular needs and application.

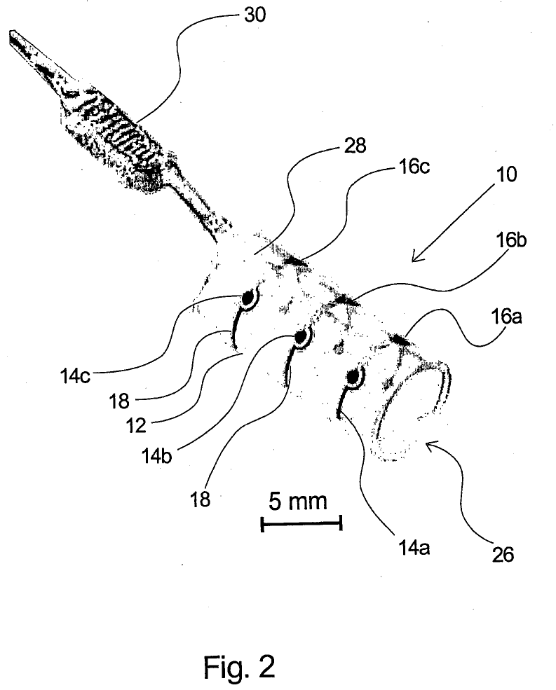

FIG. 2 shows an example of a prior art multi-polar cuff electrode fabricated by IBMT, Fraunhofer-Institute for Biomedical Technology, in St. Ingbert, Germany. Such a multi-polar cuff electrode may be used in connection with the present invention, which will be explained in the following. This cuff electrode 10 is essentially designed similarly to the cuff described above and comprises a basis or cuff part 12, which is made of a polymer. The cuff 12 may be split longitudinally as indicated by 26 or the cuff can be a spiralling or coiled structure in order to allow the cuff to be applied to a peripheral trunk nerve. A number of tripolar sets of contact sites, e.g. 14a, 14b, 14c, 16a, 16b and 16c are located on the interior wall of the cuff part 12. As indicated, four sets of contacts may be provided, distributed uniformly circumferentially around the nerve, e.g. positioned at 0°, at 90°, at 180° and at 270°.

Conductive traces 18 provided on the basis or cuff part 12 are connected to the electrode contacts and are led via a multiconductor 28 to a common connecting or interface part 30, to which external communication may be established by wires etc.

When used in connection with the invention it will be understood that one or more internal electrodes defining intrafascicular contact sites or interfascicular contact sites are introduced to the peripheral trunk nerve in question, e.g. by surgically introducing this or these internal electrodes to the trunk nerve and potentially to a nerve fascicle 4. Subsequently, a nerve cuff is applied to the trunk nerve 2, whereby the sets of surface (epineural) placed electrode contacts are located adjacent the epineurium 8 of the trunk nerve. The nerve cuff 10 is applied to the trunk nerve at the site (or sites) of internal contact sites, whereby the external and the internal contact sites are positioned at essentially the same part of the nerve trunk in question.

The connecting parts (22; FIG. 1) of the internal electrode (20; FIG. 1) may be connected or fixed to a part of the basis or cuff part 12 and/or to the common connecting or interface part 30. The internal electrode or electrodes may be connected to the nerve cuff 10 in advance or may be connected to this part after the insertion of the internal electrode(s) in an inner compartment of the trunk nerve, either before the cuff electrode 12 is applied or after. The electrical communication to and/or from the internal electrode(s) may thus be provided by means of a common multi-conductor connected to the nerve cuff electrode 12, e.g. to the common connecting or interface part 30.

The internal electrode(s), e.g. 20 may be fabricated from fine wires, e.g. a single strand of 25 μm diameter platinum-iridium wire with Teflon insulation (for example manufactured by A-M System, Inc. Carlsberg, Wash.), conductive polymers which are drawn into filaments, polymer ribbons that are overlayed with conductive traces or other electrode carriers.

When the internal electrode is manufactured from an insulated conductor, which is normally the case, the insulation has to absent or to be removed at the intended contact site. The insulation may be removed in connection with the implantation of the internal electrode(s). A contact site window may for example have a length of 1 mm in order to achieve a reliable and sufficient electrical connection.

The basis or cuff part 12 may be fabricated from a suitable polymer, e.g. silicone, and the surface (epineural) electrode contacts and the connecting traces 18 may be located on a separate substrate, e.g. a polyimide substrate located on the inner wall of the cuff part 12. Other suitable materials may be used as well.

The contact electrodes 14, 16 and/or the conductive traces 18 may be fabricated from a suitable electrically conducting material such as platinum.

Other forms of nerve cuffs than the illustrated one may be utilized, e.g. self-curling, spiral cuff electrodes etc.

The number of contact sets, e.g. 14, 16 may be varied in accordance with the circumstances. For example six sets of contacts may be used instead of four as illustrated on FIG. 1 and 2. Other number of contact sets may be used as well. Further, the contact sets need not be distributed uniformly around the trunk nerve, although this will normally be the case. Further, the contact sets may each comprise more or less than three contacts, e.g. one, two, four or more, and they need not be placed with uniform spacing in the longitudinal direction.

When an internal electrode contact 24 is active e.g. used for stimulating and/or monitoring nerve activity, it will normally be used as one pole in a two-pole combination with an external electrode contact, e.g. 14a, 14b, 14c, 16a, 16b, 16c, preferably an end contact of a contact set, e.g. a tripole contact set or with another internal electrode contact.

It will be understood that according to the invention any configuration comprising an internal and an external electrode contact will be feasible, e.g. even including a configuration comprising both inter- and intrafascicular electrodes and external electrodes comprising a variety of configurations, e.g. a mixture of two- and tripolar sets distributed with varying spacing etc.

Supporting Data

The efficacy of the novel cuff according to an embodiment of the invention has been evaluated in acute experiments performed on four anesthetized adult pigs. In these studies, a fine wire interfascicular electrode (25 μm dia. platinum-iridium with Teflon insulation) having a single contact site was drawn into the center of the radial nerve in the region of the bracial plexus using a fine needle. Then a multicontact self-coiling spiral cuff containing 18 contacts arranged in 6 sets of tripoles was installed around the nerve at the same location. This system thus formed the novel cuff.

The experiment sought to determine the extent to which individual fasicles of muscle nerve fibers that project to different muscles in the pig shoulder and arm could be activated independently by using different combinations of contacts in the novel cuff. Muscle activation was registered and quantified measuring evoked EMG using bipolar electrodes implanted into nine muscles. The monitored muscles included six muscles that abductor or extend the toes (M1,M2,M3,M4,M5,M6), the wrist extensor muscle carpi radialis (M7), and three muscles of the shoulder (M7,M8,M9).

Initially the interfascicular site was not used and each tripolar set of cuff contacts was studied successively. Thus, using the contact set at 0°, trains of electrical stimuli were presented at 25 Hz. using a ‘starcase’ or pyramid paradigm. By this is meant the duration of the pulses (or pulse width—PW) was initially 20 microsec. and then increased stepwise up to a maximum of 250 microsec. before being decreased again in mirror symmetric fashion. Once the threshold for the nerve fibers of a given muscle is exceeded, a recruitment of more and more fibers occurs as the stimulus pulse duration increases during the staircase presentation.

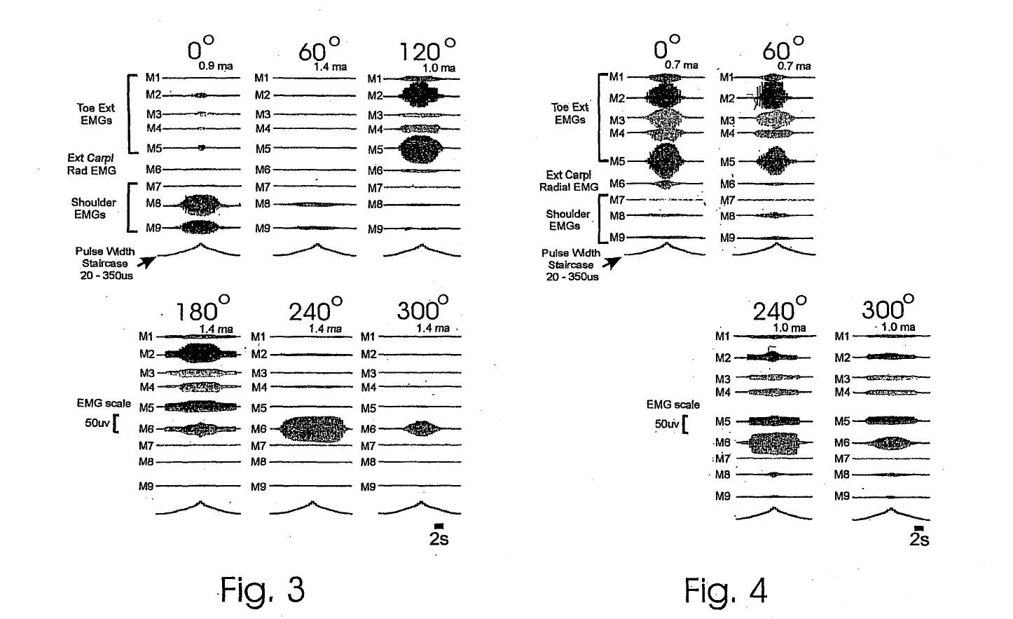

Typical results of the study using only the cuff contacts (without the interfascicular contact) are shown in FIG. 3 for one of the animals. The data shown as M1 thru M9 are the muscle EMGs developed over the course of the stimulation staircase, and the profile across all nine muscles shows the extent to which independent or ‘selective’ activation of the individual muscles was possible. For example, using the tripole set of contacts labeled 240° or 300° (FIG. 3), one can see that a pure isolated activation was possible for the M6 muscle. Also note that the use of the 0° cuff contacts activated mainly M8 and M9 while the 120° contacts activated mainly M2 and M5.

Next, the experiment with this animal was continued using the interfasicular contact in combination with each of the outer pairs of cuff contacts. The results are presented in FIG. 4. As can be seen by comparing the activation profiles of FIG. 4 with those of FIG. 3, the use of the interfascicular contact yielded different results for the four contact sets displayed in the FIG. 4. For clarity the results of using 120° and 180° for the interfascicular electrode are not shown in FIG. 4 because they were not substantially different from what was obtained with only the corresponding cuff tripoles.

What was observed with each of the four animals studied, was that at least two or three different muscle activation profiles became available when the interfascicular electrode was used versus when it was not used. For example, compare FIG. 4, 0° with FIG. 3, 180°. In each case M1,2,3,4,5, and 6 were activated, but the relative activation between M1 thur M6 was different for the FIG. 3 and FIG. 4 data. In particular, in FIG. 3, 180° M6 activation is small compared to FIG. 4, 0° whereas M6 activation is large. Such differences may become important when a muscle activation profile needs to be controlled in a Funtional Electrical Stimulation (FES) system designed to restore hand grasp in disabled persons with quadriplegia.

If additional interfascicular contact sites are provided then an even greater variety of nerve activation profiles could be expected to be obtained. Note also that although the experimental paradigm that was used studied the activation of muscle nerves within the trunk nerve, by analogy, one could expect similar selective activation of sensory nerve fascicles to occur among the sensory fascicles of a sensory nerve or of a mixed nerve.

1. Nerve electrode system for stimulating and/or monitoring at least one nerve fascicle in a trunk nerve comprising at least one internal electrode and at least one external electrode arranged in a cuff carrier system.

2. Nerve electrode system according to claim 1 characterized in that said at least one internal electrode is an intrafascicular electrode.

3. Nerve electrode system according to claim 1 or 2 characterized in that said at least one internal electrode is an interfascicular electrode.

4. Nerve electrode system according to claim 1, 2 or 3 characterized in that said at least one external electrode is a circumferential electrode attached to an inner side or substrate of said cuff carrier system.

5. Nerve electrode system according to claim 4 characterized in that the system comprises a number of external electrodes, spatially placed along the length and/or along the circumference of a trunk nerve.

6. Nerve electrode system according to one or more of claims 1-5 characterized in that said at least one internal electrode, preferably in the form of an intra- or interfascicular electrode, or a connection for said internal electrode, is connected to a base part of said cuff carrier system.

7. Nerve electrode system according to one or more of claims 1-6 characterized in that said at least one internal electrode, preferably in the form of an intra- or interfascicular electrode, comprises a conductor, e.g. in the form of a wire, a filament and/or a ribbon made of metal, electrically conducting polymer, and/or another electrically conducting material.

8. Nerve electrode system according to claim 7 characterized in that said at least one internal electrode comprises a carrier for said conductor, e.g. in the form a polymer, polymer ribbons overlayed with conductive traces or other electrode carriers.

9. Nerve electrode system according to claim 7 or 8 characterized in that said at least one internal electrode comprises means for insulation and that said insulation is locally absent or may be removed locally to provide a contact site.

10. Multi-polar nerve cuff for stimulating and/or monitoring at least one nerve fascicle in a trunk nerve characterized in that it comprises at least one internal electrode for providing an electrode site within the trunk nerve.

11. Multi-polar nerve cuff according to claim 10 characterized in that said at least one internal electrode is an intrafascicular electrode.

12. Multi-polar nerve cuff according to claim 10 or 11 characterized in that said at least one internal electrode is an interfascicular electrode.

13. Multi-polar nerve cuff according to claim 10, 11 or 12 characterized in that it comprises a number of external electrodes, spatially placed along the length and/or along the circumference of the nerve cuff.

14. Multi-polar nerve cuff according to one or more of claims 10-13 characterized in that said at least one internal electrode, preferably in the form of an intra- or interfascicular electrode, or a connection for said internal electrode, is connected to a base part of the nerve cuff.

15. Multi-polar nerve cuff according to one or more of claims 10-14 characterized in that said at least one internal electrode, preferably in the form of an intra- or interfascicular electrode, comprises a conductor, e.g. in the form of a wire, a filament and/or a ribbon made of metal, electrically conducting polymer, and/or another electrically conducting material.

16. Multi-polar nerve cuff according to claim 15 characterized in that said at least one internal electrode comprises a carrier for said conductor, e.g. in the form a polymer, polymer ribbons overlayed with conductive traces or other electrode carriers.

17. Multi-polar nerve cuff according to claim 15 or 16 characterized in that said at least one internal electrode comprises means for insulation and that said insulation is locally absent or may be removed locally to provide a contact site.

18. Method of installing a nerve electrode system according to one or more of claims 1-9 or a multi-polar nerve cuff according to one or more of claims 10-17, whereby
at least one internal electrode is introduced in a trunk nerve, after which
a basis part, e.g. a cuff of said system or nerve cuff is applied around the trunk nerve in the vicinity of the internal electrode, or vice versa.

19. Use of a nerve electrode system according to one or more of claims 1-9 or a multi-polar nerve cuff according to one or more of claims 10-17 for activation or stimulation of nerve fascicles, e.g. motor nerve fascicles.

20. Use of a nerve electrode system according to one or more of claims 1-9 or a multi-polar nerve cuff according to one or more of claims 10-17 for activation or stimulation of nerves, e.g. peripheral nerves, cranial nerves, spinal nerves etc. for restoration of sensory functions, e.g. activation or stimulation of optic nerves, auditory nerves or other sensory nerves.

21. Use of a nerve electrode system according to one or more of claims 1-9 or a multi-polar nerve cuff according to one or more of claims 10-17 for recording or monitoring of nerve fascicles, e.g. for neuroprosthesis control etc.

22. The nerve electrode system according to claim 21, wherein said at least one internal electrode is an intrafascicular electrode.

23. The nerve electrode system according to claim 21, wherein said at least one internal electrode is an interfascicular electrode.

24. The nerve electrode system according to claim 21, wherein said at least one external electrode is a circumferential electrode attached to an inner side of said cuff carrier system.

25. The nerve electrode system according to claim 24, wherein the system comprises a number of external electrodes, spatially placed along the length and/or along the circumference of a trunk nerve.

26. The nerve electrode system according to claim 21, wherein said at least one internal electrode is connected to a base part of said cuff carrier system.

27. The nerve electrode system according to claim 21, wherein said at least one internal electrode is in the form of an intra- or interfascicular electrode and comprises a conductor selected from the group consisting of a wire, a filament, a ribbon made of metal, and an electrically conducting polymer.

28. The nerve electrode system according to claim 27, wherein said at least one internal electrode comprises a carrier for said conductor, said carrier being selected from the group consisting of a polymer and a polymer ribbon overlayed with conductive traces.

29. The nerve electrode system according to claim 28, wherein said at least one internal electrode comprises means for insulation and that said insulation is locally absent or may be removed locally to provide a contact site.

30. A multi-polar nerve cuff for stimulating and/or monitoring at least one nerve fascicle in a trunk nerve wherein it comprises at least one internal electrode for providing an electrode site within the trunk nerve.

31. The multi-polar nerve cuff according to claim 30, wherein said at least one internal electrode is an intrafascicular electrode.

32. The multi-polar nerve cuff according to claim 30, wherein said at least one internal electrode is an interfascicular electrode.

33. The multi-polar nerve cuff according to claim 30, wherein it comprises a number of external electrodes, spatially placed along the length and/or along the circumference of the nerve cuff.

34. The multi-polar nerve cuff according to claim 30, wherein said at least one internal electrode is in the form of an intra- or interfascicular electrode and is connected to a base part of the nerve cuff.

35. The multi-polar nerve cuff according to claim 30, wherein said at least one internal electrode is in the form of an intra- or interfascicular electrode and comprises a conductor selected from the group consisting of a wire, a filament, a ribbon made of metal, and an electrically conducting polymer.

36. The multi-polar nerve cuff according to claim 35, wherein said conductor comprises a polymer or a polymer ribbon overlayed with conductive traces.

37. The multi-polar nerve cuff according to claim 35, wherein said at least one internal electrode comprises means for insulation and that said insulation is locally absent or may be removed locally to provide a contact site.

38. The method of installing a nerve electrode system according to claim 21, wherein at least one internal electrode is introduced in a trunk nerve, before or after which a basis part is applied around the trunk nerve in the vicinity of the internal electrode.

39. A method for activation or stimulation of nerve fascicles by installing a nerve electrode system according to claim 21, wherein at least one internal electrode is introduced in a trunk nerve, before or after which a basis part is applied around the trunk nerve in the vicinity of the internal electrode.

40. The method according to claim 39, wherein said activation is for the monitoring of nerve fascicles or for controlling a prosthesis.
